(19) United States
(12) Patent Application Publication
(10) Pub. No.: US 2025/0118417 A1
(43) Pub. Date: Apr. 10, 2025

(54) METHOD FOR PROVIDING MENTAL HEALTH ADVICE, A METHOD FOR TRAINING A DEEP-LEARNING NETWORK AND A DEEP-LEARNING BASED MENTAL HEALTH ADVISORY SYSTEM USING ACCEPTANCE AND COMMITMENT THERAPY

(71) Applicant: The Chinese University of Hong Kong, Hong Kong (CN)

(72) Inventors: Yuen Yu CHONG, Hong Kong (CN); Wai Tong CHIEN, Hong Kong (CN)

(21) Appl. No.: 18/777,770

(22) Filed: Jul. 19, 2024

Related U.S. Application Data
(60) Provisional application No. 63/587,850, filed on Oct. 4, 2023.

(51) Int. Cl. G16H 20/70; G16H 10/20; G16H 10/60; G16H 40/20; G16H 50/30; G16H 80/00

(52) U.S. Cl. CPC G16H 20/70; G16H 10/20; G16H 10/60; G16H 40/20; G16H 50/30; G16H 80/00

(57) ABSTRACT

A method for providing mental health advice, a method for training a deep-learning network and a deep-learning based mental health advisory system using Acceptance and Commitment Therapy (ACT). The method for providing mental health advice comprises the steps of: receiving textual input from a user in a consultation session; processing the textual input by applying a mental health condition relationship to the textual input to identify the mental health status of the user; and providing an output associated with the mental health status of the user.

### CROSS-REFERENCE TO RELATED APPLICATION

This application claims the benefit of U.S. Provisional Application Ser. No. 63/587,850, filed Oct. 4, 2023, which is hereby incorporated by reference in its entirety including any tables, figures, or drawings.

### TECHNICAL FIELD

This invention relates to a method for providing mental health advice and a method for training a deep-learning network in a system for providing mental health advice. Particularly, although not exclusively, the invention relates to a deep-learning based mental health advisory system using Acceptance and Commitment Therapy (ACT).

### BACKGROUND OF THE INVENTION

Hong Kong has witnessed a sharp rise in the prevalence of special needs among children. This increase, ranging from 6.2% to 10.7% annually, is evident in the growing number of preschoolers and school-aged children diagnosed with special educational needs (SEN). In 2019, there were 17,193 newly diagnosed preschool cases and 56,640 registered school-aged students with SEN during the 2020/2021 school year, reflecting a 30% increase since the 2012/14 school year. This surge can be primarily attributed to the heightened awareness and improved early identification mechanisms for children, facilitated through the deployment of on-site preschool rehabilitation services (OPRS) administered by the Social Welfare Department. The task of nurturing a young child with special needs has long been acknowledged as a demanding endeavor for parents. However, the challenges are further compounded by the protracted waiting periods endured for both diagnosis and early interventions, which can extend up to a daunting 19.6 months. This extended period of uncertainty amplifies the strain on parents, often resulting in the manifestation of symptoms related to depression and anxiety.

Securing access to effective, comprehensive, and easily obtainable mental health services presents a formidable challenge for parents in this context. A confluence of factors, including the relentless demands of caregiving, geographical impediments, and the pervasive phenomenon of self-stigma, all serve as significant barriers. The latter, in particular, is characterized by self-blame regarding one's parenting capabilities, which in turn deters parents from actively seeking face-to-face professional mental health services, often perceived as cost-prohibitive.

### SUMMARY OF THE INVENTION

The present invention provides systems and methods for rendering deep-learning-based mental health advisory services using Acceptance and Commitment Therapy (ACT) are provided.

Some key features of the system for providing such mental health advisory services include:
1) a knowledge database and a customized question bank imitating the life-contextual and problem-focused interviews between an interventionist or a counselor and the user during the initial real-person ACT counseling session;
2) an external Large Language Model (LLM);
3) the method of irrelevant handling; and
4) an artificial intelligence (AI) logic model, which is a pre-trained Natural Language Processing (NLP) model based on the Bidirectional Encoder Representations from Transformers (BERT) architecture, including the RoBERTa (Robustly Optimized BERT Pretraining Approach) architecture, that has learned the ACT counseling logic.

In accordance with the first aspect of the present invention, a method for delivering mental health advice is disclosed. This method includes the steps of receiving a textual input from a user during an ACT counseling session; processing the textual input by applying a predefined relationship on mental health conditions, thereby determining the user's mental health status, and generating an output that is associated with the identified mental health status of the user.

In accordance with the first aspect, the relationship on mental health conditions is trained by a deep-learning network.

In accordance with the first aspect, the deep-learning network is a deep-neural network arranged to identify the mental health status based on the received textual input being labeled by the deep-neural network.

In accordance with the first aspect, the textual input includes dialogues provided by the user in response to one or more predetermined questions from a question bank based on Acceptance and Commitment Therapy (ACT) knowledge, using an artificial intelligence (AI) chatbot as an interface during an ACT counseling session.

In accordance with the first aspect, the question bank is configured to imitate life-contextual and problem-focused interviews between a counselor and the user at a real-person ACT counseling session.

In accordance with the first aspect, the question bank is generated upon an initial real-person ACT counseling session between the counselor and the user.

In accordance with the first aspect, the question bank is generated based on a self-reported mental health assessment completed by the user, wherein the self-reported mental health assessment employs validated tools that evaluate a range of mental or psychological health symptoms, as well as well-being, including but not limited to anxiety symptoms, depressive symptoms, stress, and the psychological inflexibility or psychological flexibility status of the user.

In accordance with the first aspect, the method further comprises the step of providing the AI chatbot interface configured to facilitate posing one or more predetermined questions to the user and receiving textual input from the user during the ACT counseling session.

In accordance with the first aspect, one or more predetermined questions are provided to the user in the form of a questionnaire and/or one or more chat dialogues.

In accordance with the first aspect, the method further comprises the step of detecting at least one irrelevant response provided by the user when using the AI chatbot in the ACT counseling session and obtaining confirmative responses from the user.

In accordance with the first aspect, the AI chatbot interface is arranged to adjust lines of inquiry upon determination of non-pertinent user responses, if a predetermined number of irrelevant responses is detected.

In accordance with the first aspect, the AI chatbot interface is configured to administer an additional set of questions to the user upon detecting non-pertinent responses, thereby eliciting confirmatory responses essential for the accurate assessment of the user's mental health status.

In accordance with the first aspect, the AI chatbot interface is further configured to autonomously initiate and maintain engagement with the user, and to direct conversations in a manner that extracts information of greater relevance for assessing the mental health status of the user.

In accordance with the first aspect, the output includes an occurrence of irrelevancy in the ACT counseling session.

In accordance with the first aspect, the AI chatbot interface is further supported by an external Large Language Model (LLM) processing engine arranged to enhance the generation of contextually relevant responses to facilitate interaction with the user via the AI chatbot interface.

In accordance with the first aspect, the method further comprises a step involving the pre-processing of textual input before the application of a mental health condition relationship model to the textual data, aimed at accurately identifying the user's mental health status.

In accordance with the first aspect, pre-processing of the textual input includes processing the textual input with at least one of removing stopwords, lowercasing, punctuation normalizing, back translation and data augmentation.

In accordance with the first aspect, the mental health status includes psychological inflexibility and flexibility of the user.

In accordance with the first aspect, the mental health status is associated with the following processes of psychological inflexibility:
experiential avoidance;
cognitive fusion;
conceptualized past and fear of future;
attachment to conceptualized self;
lack of values clarity; and
inaction, impulsivity, avoidance persistence.

In accordance with the first aspect, the mental health status is associated with the following processes of psychological flexibility:
acceptance;
defusion;
present-moment awareness;
self-as-context;
values clarifications; and
committed action.

In accordance with the first aspect, the learning network comprises a pre-trained Natural Language Processing (NLP) model utilizing the Bidirectional Encoder Representations from Transformers (BERT) architecture, including RoBERTa (Robustly Optimized BERT Pretraining Approach) architecture, which has been specifically trained on ACT counseling logic.

In accordance with a second aspect of the present invention, a method for training a deep-learning network in accordance with the first aspect is disclosed, comprising the step of training the learning network including collecting data, pre-processing data, tokenization, model prediction, model evaluation, and optimizing hyperparameters.

In accordance with the second aspect, the method further comprises the step of fine-tuning the deep-learning network by an Adam optimizer with a cosine annealing scheduler and integrating a Retrieval-Augmented Generation (RAG) framework configured to retrieve data from a knowledge database.

In accordance with the second aspect, the knowledge database is dynamic and consistently updated with the AI chatbot user statistics and summaries of the questions asked.

In accordance with the second aspect, the deep-learning network is evaluated by assessing the diagnostic capability of a classifier to classify each process of psychological flexibility and inflexibility in ACT counseling conversation data, as a discrimination threshold is varied using the Receiver Operating Characteristic (ROC) method.

In accordance with the second aspect, the deep-learning network is further evaluated by refining the classifier's accuracy in diagnosing and identifying each process of psychological flexibility and/or inflexibility by employing a Confusion Matrix, providing a comprehensive view of the overall performance of the model and determining whether an imbalance class is mostly predicted as a major class.

In accordance with the second aspect, the method further comprises the step of testing the deep-learning network with at least one of the segmenting sentences, pre-processing data, predicting, and post-processing.

In accordance with a third aspect of the present invention, a deep-learning based mental health advisory system using Acceptance and Commitment Therapy (ACT) is disclosed, comprising: an AI chatbot interface in accordance with the first aspect provided on a user device, wherein the AI chatbot interface is supported by the deep-learning network; and a cloud service and database with a content management system (CMS).

In accordance with the third aspect, the user device is configured to collect textual input through a questionnaire and/or conversations via the AI chatbot interface and to provide functionality including scheduling appointments with counselors and facilitating interactive actions for mental health services.

In accordance with the third aspect, the system further comprises an operator interface supported by the Cloud Service and database with the CMS, wherein the operator interface is designed to facilitate the importation of data and enable the visualization of analyses pertaining to the mental health status of the user, as well as a history of chats.

In accordance with the third aspect, the system further comprises a portal designed to display the outputs associated with the mental health status of the user and/or statistics concerning detected irrelevant contents, including the frequencies and occurrences of irrelevancy.

Embodiments of the subject invention can be used for providing digital, personalized deep-learning based mental health advice using Acceptance and Commitment Therapy (ACT), imitating a real-person, therapist-led ACT session.

In one example embodiment, the method for providing mental health advice may comprise providing counseling based on Acceptance and Commitment Therapy (ACT) knowledge, by a chatbot logics module; and performing classification and analysis of mental health conditions of a user, by a categorization and analysis module. The infrastructure of the chatbot logics module comprises four components. The first component is a knowledge database and a customized question bank. The second component is an external Large Language Model (LLM). The third component is the method of irrelevant handling. The fourth component is an artificial intelligence (AI) logic model. In the categorization and analysis module, the performance of classification and analysis of the mental health conditions of a user comprises a step of training and a step of system testing. Moreover, the step of training comprises collecting data, pre-processing data, tokenization, model prediction, model evaluation, and optimizing hyperparameters. The step of pre-processing data comprises removing stopwords, lowercasing, punctuation normalizing, backtranslation and data augmentation. The step of model prediction is performed by Bidirectional Encoder Representations from Transformers (BERT) type of pre-trained model, including the RoBERTa (Robustly Optimized BERT Pretraining Approach) architecture. The method may further comprise finetuning the pre-trained model by an Adam optimizer with a cosine annealing scheduler. In addition, the step of model prediction is further enhanced by integrating a Retrieval-Augmented Generation (RAG) framework, which is designed to enrich the system's response generation capabilities by utilizing a dynamic Knowledge Database that supports the RAG functionality. The model evaluation comprises assessing the diagnostic ability of a classifier as a discrimination threshold is varied by a Receiver Operating Characteristic (ROC) method. The model evaluation also comprises providing Area under the ROC Curve (AUC) scores to quantify the area under the ROC curve which summarizes the overall performance of the classifier across all possible area/threshold settings. To further refine the classifier's accuracy in identifying psychological flexibility and/or inflexibility processes, a Confusion Matrix is employed. The analysis also discusses the granularity of the target variable in the classification task with a design of classification among three categories, and classification among six categories. Further, the system testing comprises segmenting sentences, pre-processing data, predicting, and post-processing, in which the customized segmentation method is performed to generate multiple potential paragraphs for prediction, and lastly applying means of non-maximum suppression to reduce the total number of predictions.

In another embodiment of the subject invention, a computer program product is provided. It comprises a non-transitory computer-executable storage device having computer-readable program instructions embodied thereon, which, when executed by a computer, causes the computer to perform a mental health advising method. The computer-executable program instruction comprises providing counseling based on Acceptance and Commitment Therapy (ACT) knowledge, by a chatbot logic module; and performing classification and analysis of mental health conditions of a user, by a categorization and analysis module. The infrastructure of the chatbot logics module comprises four components. The first component is a knowledge database and a customized question bank. The second component is an external Large Language Model (LLM). The third component is the method of irrelevant handling. The fourth component is an artificial intelligence (AI) logic model. In the categorization and analysis module, the performance of classification and analysis of the mental health conditions of a user comprises a step of training and a step of system testing. Moreover, the step of training comprises collecting data, pre-processing data, tokenization, model prediction, model evaluation, and optimizing hyperparameters. The step of pre-processing data comprises removing stopwords, lowercasing, punctuation normalizing, backtranslation and data augmentation. The step of model prediction is performed by Bidirectional Encoder Representations from Transformers (BERT) type of pre-trained model, including the RoBERTa (Robustly Optimized BERT Pretraining Approach) architecture. The method may further comprise finetuning the pre-trained model by an Adam optimizer with a cosine annealing scheduler. In addition, the step of model prediction is further enhanced by integrating a Retrieval-Augmented Generation (RAG) framework, which is designed to enrich the system's response generation capabilities by utilizing a dynamic Knowledge Database that supports the RAG functionality. The model evaluation comprises assessing the diagnostic ability of a classifier as a discrimination threshold is varied by a Receiver Operating Characteristic (ROC) method. The model evaluation also comprises providing Area under the ROC Curve (AUC) scores to quantify the area under the ROC curve which summarizes the overall performance of the classifier across all possible area/threshold settings. To further refine the classifier's accuracy in identifying psychological flexibility and/or inflexibility processes, a Confusion Matrix is employed. The analysis also discusses the granularity of the target variable in the classification task with a design of classification among three categories, and classification among six categories. Further, the system testing comprises segmenting sentences, pre-processing data, predicting, and post-processing, in which the customized segmentation method is performed to generate multiple potential paragraphs for prediction, and lastly applying means of non-maximum suppression to reduce the total number of predictions.

According to another embodiment of the subject invention, a method for providing mental health advice is provided. The method comprises providing counseling based on Acceptance and Commitment Therapy (ACT) knowledge, by a chatbot logics module; and performing classification and analysis of mental health conditions of a user, by a categorization and analysis module, wherein the chatbot logics module comprises four components including a knowledge database associated with a customized question bank, an external Large Language Model (LLM), a unit of irrelevant handling, and an artificial intelligence (AI) logic model. The performing classification and analysis of the mental health conditions of a user comprises a step of training and a step of system testing. The step of training comprises collecting data, pre-processing data, tokenization, model prediction, model evaluation, and optimizing hyperparameters. The step of model prediction comprises integrating a Retrieval-Augmented Generation (RAG) framework to enrich response generation capabilities based on the knowledge database configured to support the RAG framework. Moreover, the knowledge database is dynamic and consistently updated by an AI chatbot with user/chat statistics and questions summary and the question bank is customized and configured for imitating life-contextual and problem-focused interviews between an interventionist or a counselor and the user at an initial real-person ACT counseling session. The external Large Language Model (LLM) is configured to be coupled to OpenAI services to enhance the generation of contextually relevant responses when the user interacts with an AI chatbot. The external LLM could be GPT-3.5-turbo, GPT-4 and/or GPT-4 Turbo. The OpenAI services are Azure OpenAI services. In addition, the LLM is configured for processing user inputs and generating corresponding responses. The framework of the chatbot logics module is integrated into a user interface. The unit of irrelevant handling is configured to adjust lines of inquiry in case of non-pertinent user responses. Further, the unit of irrelevant handling is configured for autonomously initiating and sustaining engagement with the user and steering conversations to extract information of greater relevance for enriching downstream processes. The downstream processes include a detection task. The artificial intelligence (AI) logic model is configured for logical controlling of chatbot question flows, detecting the intention of user input, and serving as a core to integrate the four components of the chatbot logics module to perform various methods. The various methods include input and output parsing, data querying and updating, applying Retrieval-Augmented Generation (RAG), applying few-shot learning and chain-of-thought to a stream of data. The method may further comprise displaying statistics with respect to query-response pairs and the proclivity of different users towards irrelevant responses. The method may further comprise providing a snapshot of current user metrics and the quality of ongoing conversation sessions for insights and optimization of LLM applications within the ACT framework. The unit of irrelevant handling is configured for actively prompting questions for more relevant information, displaying statistics on a portal, and analyzing the performance of accuracy and success rate.

According to another embodiment of the subject invention, a Pai.ACT system for providing mental health advice is provided. The system comprises an input module configured to receive user input from chatbot artificial intelligence (AI) or a service session; a Retrieval-Augmented Generation (RAG) unit configured to retrieve from knowledge database; a chain of thought unit configured to provide prompts having intermediate steps with instructions; a few-shot learning unit configured to provide demonstrations pair with respect to target input/output template for extra information; an output parse unit configured to parse unstructured output from Large Language Model (LLM) completion into content understandable by the Pai.ACT system; an irrelevant handling unit configured to determine if a number of irrelevant detected is within a threshold, then make the AI chatbot ask another round of questions to gather more information if the result of the determination is true; and a portal unit configured to display statistics with respect to detected irrelevant contents and frequency/occurrences of irrelevancy.

According to another embodiment of the subject invention, a method for assessing the performance of a Pai.ACT system for providing mental health advice is provided. The method comprises receiving input from a user; performing labeling to generate metrics on accuracy; performing chatbot artificial intelligence (AI) to generate metrics on success rates; and performing prompt comparison and refinement.

### BRIEF DESCRIPTION OF THE DRAWINGS

FIG. 1 is a schematic diagram of a computer server which is arranged to be implemented as a processor of a mental health advisory system in accordance with an embodiment of the present invention.

FIG. 2 is a block diagram showing a mental health advisory system in accordance with an embodiment of the present invention.

FIG. 3 is a block diagram showing a mental health advisory system in accordance with an alternative embodiment of the present invention, in which the mental health advisory system is also referred as Pai.ACT when being implemented as an application which may be run on a user device.

FIG. 4 is a radar chart showing psychological flexibility or inflexibility processes and associated pillars of these processes in Acceptance and Commitment Therapy, displayed as an output associated with the mental health status of the user in accordance with an embodiment of the present invention.

FIG. 5 is a graphical plot of Receiver Operating Characteristic (ROC) that illustrates the diagnostic ability of a classifier of six processes of psychological inflexibility including experiential avoidance, cognitive fusion, attachment to the conceptualized self, lack of present-moment awareness, and inaction/impulsivity in the pursuit of the valued goal as its discrimination threshold is varied, according to an embodiment of the present invention.

FIG. 6 is a graphical plot of Receiver Operating Characteristic (ROC) that illustrates the diagnostic ability of a classifier of three pillars of psychological inflexibility including emotional avoidance, rule-following and behavioral avoidance as its discrimination threshold is varied, according to an embodiment of the present invention.

FIG. 7 is a graphical plot of the AUC score, which quantifies the area under the ROC curve and summarizes the overall performance of the classifier across all possible area/threshold settings.

FIG. 8 is a block diagram illustrating a deep-learning based mental health advisory system using Acceptance and Commitment Therapy (ACT) in accordance with an embodiment of the present invention.

### DETAILED DISCLOSURE OF THE INVENTION

According to the embodiments of the present invention, an example of an artificial intelligence-driven, chatbot-assisted mental health method and systems possessing the domain knowledge in Acceptance and Commitment Therapy (ACT, an evidence-based low-intense psychotherapy) provided to furnish mental health services for users, such as, but not limited to parents of children with special needs, or caregivers of others such as elderly or teenagers is herein described.

The terminology used herein is for the purpose of describing particular embodiments only and is not intended to be limiting to the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

When the term “about” is used herein, in conjunction with a numerical value, it is understood that the value can be in a range of 90% of the value to 110% of the value, i.e. the value can be +/−10% of the stated value. For example, “about 1 kg” means from 0.90 kg to 1.1 kg.

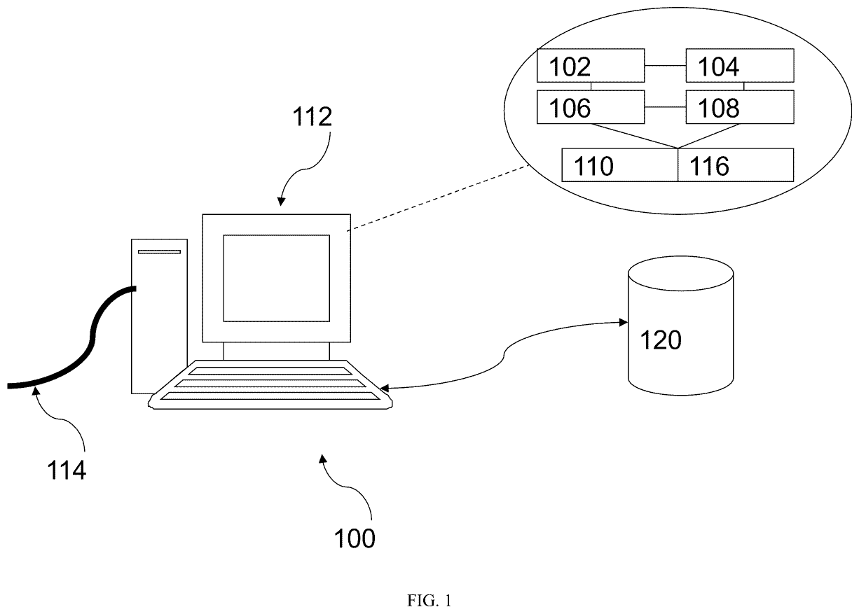

Referring to FIG. 1, an embodiment of the present invention is illustrated. This embodiment is arranged to provide a system for providing mental health advice, by performing a method comprising the steps of: receiving a textual input from a user in a consultation session; processing the textual input by applying a mental health condition relationship to the textual input so as to identify a mental health status of the user; and providing an output associated with the mental health status of the user.

In this example embodiment, the interface and processor are implemented by a computer having an appropriate user interface. The computer may be implemented by any computing architecture, including portable computers, tablet computers, stand-alone Personal Computers (PCs), smart devices, Internet of Things (IoT) devices, edge computing devices, client/server architecture, “dumb” terminal/mainframe architecture, cloud-computing based architecture, or any other appropriate architecture. The computing device may be appropriately programmed to implement the invention.

The system may be used to receive textual input from a user, such as a plurality of responses provided by a user in a conversation session. In particular, the user may be arranged to answer a list of questions that are useful for a mental health counselor or practitioner to understand the mental health status of the user according to the response provided by the user. The responses may then be analyzed by the system to predict or determine the mental health status, similar to how the mental health counselor or practitioner would perform or act in a real-person counseling session, and the system may further provide an output such as a report or advice to the user so as to let the user understand or at least learn more of the user's mental health status. In this disclosure, a counseling session generally refers to an Acceptance and Commitment Therapy (ACT) counseling session, when ACT counselling is provided to the user.

As shown in FIG. 1, a schematic diagram of a computer system or server, labeled 100, is presented. This diagram represents an example embodiment of a processor within the health monitoring system. In this embodiment, the system comprises a server 100 which includes suitable components necessary to receive, store and execute appropriate computer instructions. The components may include a processing unit 102, including Central Processing Unit (CPUs), Math Co-Processing Unit (Math Processor), Graphic Processing Unit (GPUs) or Tensor processing united (TPUs) for tensor or multi-dimensional array calculations or manipulation operations, read-only memory (ROM) 104, random access memory (RAM) 106, and input/output devices such as disk drives 108, input devices 110 such as an Ethernet port, a USB port, etc. Display 112 such as a liquid crystal display, a light emitting display, or any other suitable display and communications links 114. The server 100 may include instructions that may be included in ROM 104, RAM 106 or disk drives 108 and may be executed by the processing unit 102. There may be provided a plurality of communication links 114 which may variously connect to one or more computing devices such as a server, personal computers, terminals, wireless or handheld computing devices, Internet of Things (IoT) devices, smart devices, edge computing devices, cloud devices. At least one of a plurality of communications links may be connected to an external computing network through a telephone line or other type of communications link.

The server 100 may include storage devices such as a disk drive 108 which may encompass solid state drives, hard disk drives, optical drives, magnetic tape drives or remote or cloud-based storage devices. The server 100 may use a single disk drive or multiple disk drives, or a remote storage service 120. The server 100 may also have a suitable operating system 116 which resides on the disk drive or in the ROM of the server 100.

The computer or computing apparatus may also provide the necessary computational capabilities to operate or to interface with a machine learning network, such as neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

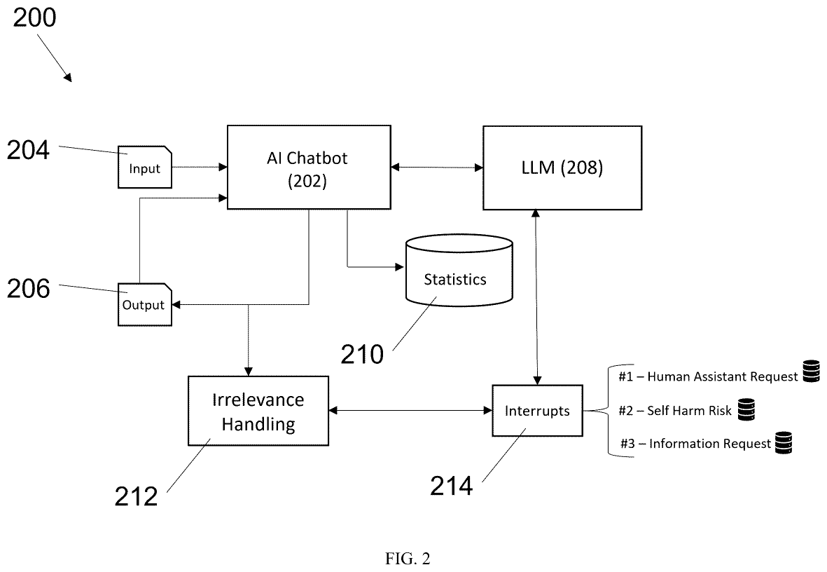

With reference to FIG. 2, an embodiment of the health monitoring system, labeled 200, is shown. In this embodiment, the server 100 is used as part of system 200, which is structured to receive textual input from the user, process this input through the application of a mental health condition relationship, and generate an output concerning the user's mental health status directly provided to the user or may alternatively be reviewed by a medical professional, such as a doctor or psychologist.

For example, the system 200 may be used for providing mental health advice or counseling to parents of children with special educational needs (SEN). These parents can interact with the system, e.g. by “chatting” with the AI chatbot in the system using a voice-to-text function embedded in a mobile application, which simulates an initial ACT counseling session, during which the system poses a curated list of questions to the parents to elicit their responses. Based on these interactions, the mental health status of the parents, characterized by measures of psychological flexibility or inflexibility, can be assessed. Consequently, this enables the parents to gain a deeper understanding of their psychological state and consider whether to pursue any further consultation or therapy.

Alternatively, the system may also be utilized to provide mental health advice or counseling to other groups, such as caregivers of the elderly or teenagers. In these scenarios, the system can offer tailored questions that better align with the specific situations or conditions of these users. Ideally, the system has access to an array of knowledge databases centered on ACT, facilitating the replication of the contextual and problem-focused interviews typically conducted in initial ACT sessions between an ACT interventionist or counselor and the user. These sessions are tailored for various caregiver groups. Detailed descriptions of the system's various applications will be provided subsequently in this disclosure.

Preferably, the mental health condition relationship, applied to textual inputs from users to ascertain their mental health status, is optimized through a learning network, such as a deep neural network. This network is specifically trained to determine mental health status based on the textual inputs, which are appropriately labeled by the deep neural network. In one preferred embodiment, the textual inputs consist of dialogues provided by users in response to one or more predetermined questions which are sourced from a question bank that is grounded in Acceptance and Commitment Therapy (ACT) principles, utilized during the counseling sessions.

Without wishing to be bound by theory, Acceptance and Commitment Therapy (ACT) is an evidence-based low-intense psychotherapy that stems from traditional behavior therapy and cognitive behavioral therapy. Acceptance and Commitment Therapy (ACT) is designed to help individuals not merely cease the avoidance, denial, and struggle with their internal emotions, but rather embrace and acknowledge these profound emotional responses as natural reactions to specific situations. This understanding should not impede their progress in life. With this awareness, individuals are guided to accept their challenges and commit to necessary behavioral changes that align with their values, meaning, and purpose in life, regardless of their current life circumstances and emotional states.

Acceptance and Commitment Therapy (ACT) additionally promotes a heightened commitment to healthy, constructive activities that support personal values or goals. It aims to assist individuals in moving through challenging emotions, thereby redirecting energy towards healing rather than lingering on negative experiences. Research has demonstrated that ACT can effectively address a range of mental and physical conditions, including anxiety disorders, depression, obsessive-compulsive disorder, psychosis, eating disorders, substance use disorders, workplace stress, and chronic pain.

The six processes of psychological inflexibility, which reflect an individual's mental health status and can lead to increased suffering and ineffective action, necessitate therapeutic intervention such as ACT to transition from psychological inflexibility to flexibility. These processes are explained as follows:
Experiential avoidance: This is characterized by an unwillingness to remain in contact with uncomfortable thoughts, feelings, memories, and physical sensations, leading to attempts to suppress, deny, or escape these experiences.
Cognitive fusion: This involves becoming entangled with our thoughts and feelings so that they dominate our behavior, often in ways inconsistent with our underlying values.
Conceptualized past and fear of the future: This process involves excessive attachment to a rigid identity based on past experiences or relentless anxiety about potential future scenarios, overshadowing the present moment. Being overly fixated on the past and future contrasts with being present-oriented.
Attachment to conceptualized self: This is identifying strictly with the self-concept (the story one tells oneself about oneself) rather than understanding the self as a context-a perspective from which experiences are observed. This can limit perspective and flexibility in responding to changing circumstances.
Lack of values clarity: Unlike the clear understanding and commitment to personal values seen in psychological flexibility, psychological inflexibility involves unclear or uncommitted values, which can lead to aimless or unfulfilling patterns of behavior.
Inaction, impulsivity, avoidance persistence: This refers to behaviors that do not align with chosen values, whether through failure to act, acting without consideration of long-term impact, or persistently engaging in behaviors that avoid discomfort at the expense of personal growth or achievement.

The six processes of psychological flexibility, which serve as indicators of an individual's mental health, are essential components of ACT. These processes facilitate improved mental health and adaptive functioning. The processes are explained as follows:
Acceptance: This process involves acknowledging and embracing all thoughts and emotions without attempting to avoid, deny, or change them. Acceptance is not about passivity or resignation but about recognizing our inner experiences as they are, which can reduce the struggle associated with resistance.
Cognitive defusion: Cognitive defusion techniques teach individuals to detach from the content of their thoughts and see them as merely words or pictures, rather than what they literally assert. This helps reduce their impact and influence over behavior and emotions.
Present moment awareness: This emphasizes active engagement with the present moment. It encourages mindfulness and an open, receptive, and non-judgmental stance toward current experiences, allowing individuals to disengage from automatic thoughts about the past or future.
Self-as-context: This principle assists individuals in understanding that they are more than their thoughts and feelings. By adopting the perspective of ‘Self as Context,’ individuals learn to observe their experiences without attachment to self.
Values clarifications: This focuses on clarifying what is most important to the individual, essentially defining personal values that guide and motivate behavior. Values act as a compass that helps individuals direct their actions in meaningful ways.
Commitment to action: This involves making changes in behavior that are aligned with the identified values. Commitment is about taking effective action that leads to a rich, full, and meaningful life, even when it involves moving through difficult or uncomfortable experiences.

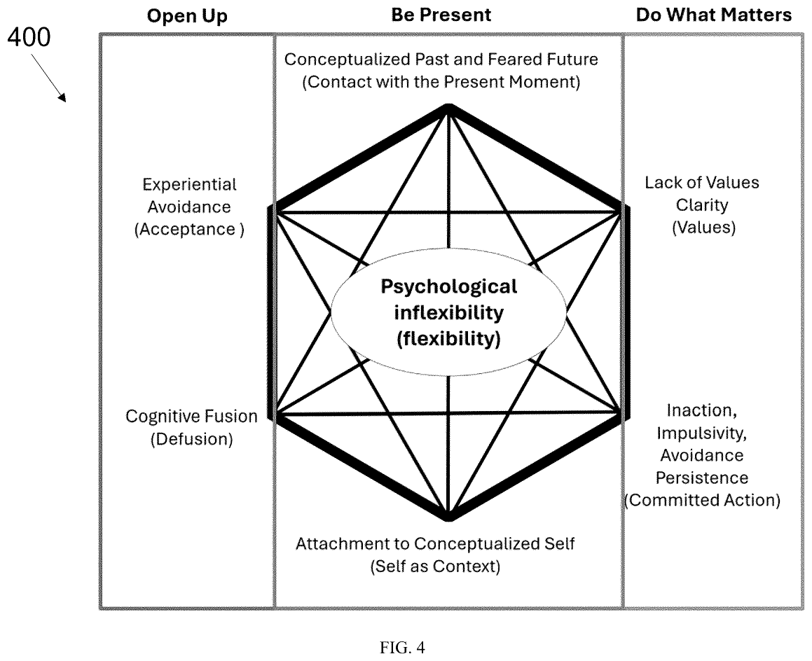

In these example embodiments, psychological flexibility and inflexibility processes may be referred to with different terms, e.g. as further illustrated in the example radar chart in FIG. 4.

In one example embodiment, the method of the subject invention is developed based on the analysis of over 18,000 transcript-based texts illustrating the individual, video-conferencing-based ACT counseling sessions between parents and experienced counselors. In this example, the transcript-based texts and the associated analysis provided by ACT counselors are included in the knowledge database for training the classifier arranged to classify/categorize responses provided by a user of the system including the learning network.

More preferably, the learning network may be a pre-trained Natural Language Processing (NLP) model based on Bidirectional Encoder Representations from Transformers (BERT) architecture, including the RoBERTa (Robustly Optimized BERT Pretraining Approach) architecture, that has learned ACT counseling logic. Using the BERT-based model for natural language processing (NLP) tasks, the analysis has led to the creation of the pre-trained deep-learning language model of the method and systems of the subject invention that can accurately identify texts and their context of different languages/dialects, and by learning the counseling logics of the ACT, i.e. training the learning network with the ACT counseling logic, psychological (in)flexibility processes of users may be accurately identified by the system.

For example, the training database may be a Hong Kong-centric, Cantonese/Chinese-specific ACT database catering to parents of children with special needs developed through the method and systems of the subject invention. Beyond capturing dialogues between the ACT experts and the parents, this database also incorporates expert-led feature engineering through the platform, comprising (1) spotlighting keywords within the conversations, (2) annotating ACT category principles to construct an ACT case conceptualization, (3) discerning specific conversation keywords indicative of mental health severity, and (4) determining appropriate stepped-care, ACT-specific mental health interventions. Collectively, these steps foster an all-encompassing learning process with the deep-learning method, based on the intricacies of the ACT counseling logics.

It should be appreciated by a skilled person in the art that, a different training database may be used for training a learning network, preferably a deep-learning network, based on a different target language/dialect if necessary, and it is also possible that a learning network based on a different language may be obtained if a suitable model transformation or adaptation process is employed. As an example, where the system may be deployed for use in providing mental health advice or counseling to teenagers, a different corpus, based on the conversations or dialogues between teenage patients and their counselors or health care providers may be used as the training corpus in building a new mental health condition relationship which can be used by the system. The corpus may be labeled or otherwise processed by experts or with supervised, semi-supervised or unsupervised learning techniques and then be used by the system to provide the necessary mental health advice or counseling to the targeted groups. Other groups that may benefit from the system may include, without limitations, elderly persons, children, students, teachers, workers, disaster relief workers and victims, emergency workers, law enforcement personnel, military personnel, medical staff, social workers, unemployed persons, patients, socially vulnerable groups, disadvantage groups, displaced groups, minority groups, prisoners or any other groups that require mental health advice or counseling.

This pre-trained deep-learning language model of the method and systems of the subject invention may accurately identify and classify the user's psychological flexibility and/or inflexibility process(es) (e.g. experiential avoidance, cognitive fusion, attachment to the conceptualized self, lack of present-moment awareness, and inaction/impulsivity in the pursuit of valued goal) and/or three pillars of flexibility and/or three pillars of inflexibility processes including emotional avoidance, rule-following, behavioral avoidance in dialogue texts in a consultation session.

As described earlier, users of the system may “chat” with the system similar to chatting with a real counselor. With reference to FIG. 2, a chatbot interface, hereinafter also referred as “AI chatbot” 202 may be provided to facilitate asking the user with one or more predetermined questions and receiving the textual input 204 from the user in the ACT counseling session. In this disclosure, a counseling session may broadly mean a period when a user seeks mental health advice or uses the system for providing mental health advice in accordance with the embodiments of the present invention. In addition, the AI chatbot interface 202 also facilitates the display of various information to the user and a medical professional or counselor (also a user of the system, but from the view of assisting the patient user), such as the questions as well as the analysis results or the output 206 associated with the mental health status of the user.

Preferably, an AI chatbot interface, designated as 202, can be implemented on a user device, such as a mobile application executable on devices including smartphones or tablet computers. The AI chatbot interface 202 is backed by a supportive learning network, enabling users to interact with the system 200 as if they are conversing with an actual counselor which mimics human-like interactions through the mobile application. Additionally, a cloud service and/or database might be accessible via the mobile application on the user device in certain exemplary embodiments. For example, the database and potentially segments of the model may be dynamically updated and provided as a cloud service, allowing the mobile application to remain lightweight.

In one preferred embodiment, the AI chatbot interface 202 is further supported by an external Large Language Model (LLM) processing engine 208, or a natural language processing (NLP) engine, arranged to enhance the generation of contextually relevant responses to facilitate interacting with the user via the AI chatbot interface.

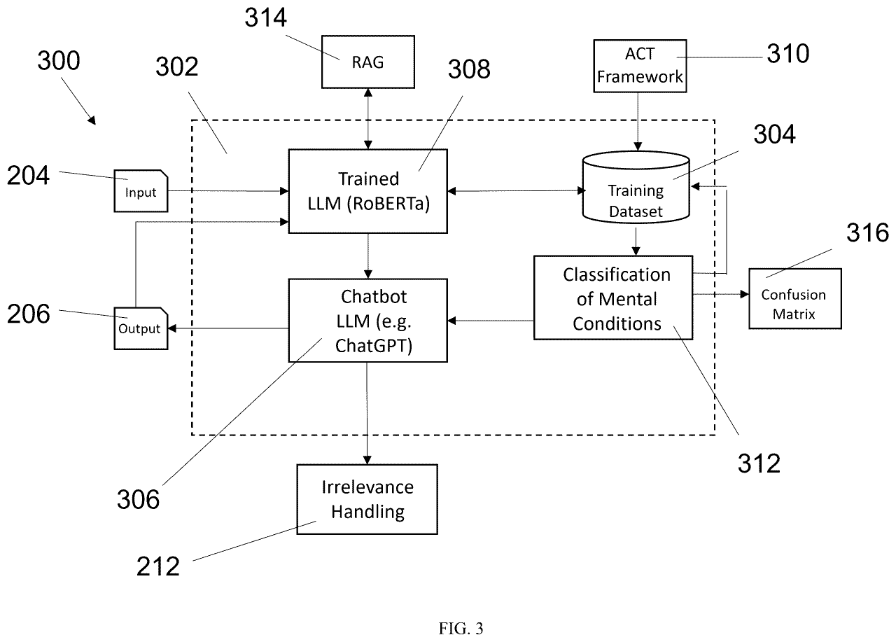

For example, with reference also to FIG. 3, in one embodiment, the method and systems of the subject invention are configured to integrate the AI-driven ACT logic model with a third-party LLM/NLP model such as OpenAI, which may be employed to answer general questions and provide empathic responses. Moreover, the method and systems of the subject invention include a voice-to-text chatbot module that imitates human responses from trained ACT counselors, allowing the users to engage in empathetic conversations and receive personalized mental health interventions including assessments, consultations, process-matched ACT intervention modules using texts, audio records and/or video animations and tailored stepped-care interventions based on the ACT principles through a mobile application 300 named as ‘Pai.ACT’ in one preferred embodiment.

In this example, the user device, with Pai.ACT 300 installed or running on the user device, can collect the textual input 204 through a questionnaire and/or conversations via the AI chatbot interface 202, which may be further processed for determining the mental health status of the user. In addition, Pai.ACT 300 may also provide functionality including scheduling appointments with counselors and facilitating interactive actions for mental health services. For instance, should the need for more intensive mental health support be identified by the Pai.ACT application 300, users may arrange to meet with ACT counselors in real-time. Moreover, Pai.ACT can deliver questions commonly utilized in ACT therapy to the user in the form of a questionnaire and/or through one or more chat dialogues.

In one example embodiment, the questions or the question bank used in the AI chatbot can be generated, customized or enhanced following the completion of an initialization step. This step could occur, for instance, after a user engages with the AI chatbot, which simulates an initial ACT counseling session, or after a user completes a real-person ACT counseling session, utilizing the data from this conversation to refine the questions or question bank. Alternatively, the initialization may involve a self-reported assessment that evaluates symptoms of anxiety, depression, stress and psychological inflexibility or psychological flexibility. Subsequent consultation sessions are thus tailored specifically for the user, with customized questions provided to different users who may have varying experiences in their caregiving roles in real-life scenarios. This customization ensures that the therapeutic interactions are relevant and sensitive to the individual circumstances of each user.

The deep-learning-based method and systems of the subject invention play a dual role, functioning as an instructive platform for ACT learners all around the world while also serving as a foundational resource for learners to understand the real-life counseling principles of the ACT. The method and systems of the subject invention are pivotal in facilitating a thorough understanding of the therapeutic approach, while concurrently serving as an influential platform for nurturing a growing community of ACT experts. Through adeptly forging a seamless connection between theoretical knowledge and its pragmatic implementation, the method and systems effectively address a critical void within the training continuum. Consequently, they lay the foundation for the cultivation of a highly proficient cohort of ACT practitioners.

In a preferred embodiment as shown in FIG. 3, the mental health advisory system 300 comprises an AI chatbot logics module 302 which may include a plurality of components. The first component is a knowledge database 304 that is dynamically and consistently updated by the statistics 210 which are collected when the user interacts with the AI chatbot interface 202 (referring to FIG. 2), questions summary, as well as a customized question bank specialized in imitating the life-contextual and problem-focused interviews between an interventionist or a counselor and the user at the initial real-person ACT counseling session.

As described earlier, a transcript arising from an interaction between an interventionist or a counselor and a patient in an ACT counseling session (i.e. a corpus) can be used as the (initial) knowledge database for building the AI chatbot logics module 300. It should be appreciated by a skilled person in the art that the knowledge database 304 can be substituted with any relevant corpus depending on its application, including the early and late stages of ACT counseling. For example, the training corpus can be tailored to specific demographics or conditions, such as ACT counseling for elderly care, teenager care, chronic illness management or general population care, varying according to different applications.

The second component is an external Large Language Model (LLM) 306, for example, GPT-3.5-turbo, GPT-4 and/or GPT-4 Turbo, in conjunction with Azure OpenAI services to enhance the generation of contextually relevant responses when the user interacts with the AI chatbot 302. This model is responsible for processing user inputs and generating corresponding responses, central to the language-based interaction within the application. In addition, the framework of the chatbot logics module can be seamlessly integrated into a user interface such as a mobile application executable by a user device. Moreover, facilitated by the AI chatbot interface 202, users, such as, but not limited to, Chinese-speaking parents of children with special needs, can also utilize the voice-to-text feature to communicate their mental health needs while engaging with the chatbot-based ACT counseling logic model.

Alternatively, an external LLM 306 provided by other service providers may also be utilized for improving the AI chatbot interface 202 or text processing function This enhancement could significantly improve the user experience, making interactions with the AI-based counselor more human-like. Additionally, it is conceivable that at least part of the LLM could be deployed locally, while remaining entirely external in some example embodiments. This flexibility allows for tailored integration depending on specific implementation needs or requirements.

In addition, the language model 306 may also perform pre-processing of the textual input prior to applying the mental health condition relationship to the textual input (i.e. classifying the textual input in accordance with the processes of psychological inflexibility and/or flexibility) for identifying the mental health status of the user. It is possible that users may provide responses with incorrect or inaccurate wordings, e.g. typewritings with typographic or grammatical error, pre-processing the textual input, such as removing stopwords, lowercasing, punctuation normalizing, back translation and data augmentation may help increasing the accuracy of the determination of mental health condition/status based on classification of the pre-processed textual input.

The third component is irrelevant handling 212 wherein the AI chatbot demonstrates an enhanced ability to adjust its line of inquiry in the face of non-pertinent user responses. Preferably, it is capable of autonomously initiating and sustaining engagement with users, steering conversations to extract information of greater relevance, thereby enriching downstream processes such as detection tasks or classification of textual input.

For example, upon detecting at least one irrelevant response from the user during an ACT counseling session in which the user interacts with the AI chatbot, such a detected interrupt 214 may trigger irrelevant handing 212 where confirmative responses from the user will be obtained before the responses provided by the user are further processed, i.e. classification or labelling. In the process of irrelevant handling, the AI chatbot interface 202 may adjust lines of inquiry upon determination of non-pertinent user responses. For instance, an additional round of questions may be provided to the user if a predetermined number of irrelevant responses is detected.

In an alternative example, the AI chatbot interface 202 may autonomously initiate and sustain engagement with the user and steer conversations to extract information of greater relevance for identifying the mental health status of the user, when it is determined that the user may have lost focus or is unable to provide responses that are relevant to the ACT therapy or mental health advice being sought.

In addition, the chatbot interface 202 or the LLM 208 may detect other critical interruptions, such as indications of self-harm risk, which would immediately prompt a request for human intervention from the operator or counselor. Alternatively, it may issue a warning to the user advising immediate consultation with a medical practitioner or psychologist. In scenarios where the chatbot interface 202 or the LLM 208 determines that additional or confirmatory information is required, these interruptions will trigger further interrupt handling processes as necessary.

In one exemplary embodiment, there may be multiple architectural components and the sequence of processes within Pai.ACT 300, tracing the path from user input to the ultimate output. The architecture is crafted to enhance the functionality and responsiveness of the AI chatbot. It is particularly tailored to optimize the use of a trained LLM 308 and to manage irrelevant responses effectively during interactions with the AI chatbot, preferably incorporating the capability of irrelevant handling 212 as described earlier.

Preferably, the processing of the textual input 204 may comprise the following steps:
(i) User Inputs: This step represents the initial phase where textual data is gathered either through interactions with the AI chatbot (for example, when parents use the AI chatbot in Pai.ACT) or during service sessions (for example, when parents receive the existing ACT counseling service).
(ii) Retrieval-Augmented Generation (RAG 314): At this juncture, information is sourced from a knowledge base using identifiers such as user_id and conversation_id. Its role is to supply contextually pertinent information that will inform the ensuing response.
(iii) Chain of Thought: Here, the system employs a structured approach, utilizing prompts that provide intermediate steps with directives such that the LLM is navigated through a logical sequence of thoughts.
(iv) Few-Shot Learning (FSL): This step relies on demonstration pairs that furnish the model with explicit examples of the target input-output relationship. It proves invaluable for training models to decipher nuanced or domain-specific prompts. Within the therapeutic ambit of Pai.ACT, certain scenarios or challenges might arise infrequently but are nonetheless critical. Using few-shot learning, Pai.ACT is capable of adeptly handling such rare instances with a limited number of examples. This step also enables Pai.ACT to tailor its responses based on a concise series of interactions, fostering more personalized user experiences.
(v) Large Language Model (LLM): This step involves the primary language model processing the data and formulating a response.
(vi) Output Parsing: Following response generation by the LLM, this step involves parsing the unstructured output into structured data such that the system can interpret and utilize it effectively.
(vii) Irrelevant Handling: Should the system identify irrelevant responses according to a predefined threshold, this step triggers the chatbot to request further clarification or additional information from the user.
(viii) AI chatbot: This step may represent the overarching system that coordinates the aforementioned modules, ensuring a seamless data flow and operation continuity.
(ix) Portal: This step displays an interface, such as a user dashboard, that provides statistics pertinent to the chatbot's performance, with an emphasis on the management of irrelevant responses.

Moreover, the system of the subject invention includes a comprehensive data visualization module, capable of displaying statistics concerning query-response pairs and the proclivity of different users towards irrelevant responses. For example, a portal may be provided for displaying the output 206 associated with the mental health status of the user and/or statistics with respect to detected irrelevant contents and frequency/occurrences of irrelevancy. Hence, the system operators may be provided with a clear snapshot of current user metrics and the quality of ongoing conversation sessions with the AI chatbot. In addition, an operator interface supported by the Cloud Service and the database with the CMS may be provided to facilitate importing data and visualizing analysis of the mental health status of the user and a chat history. Advantageously, such analytical capabilities not only empower operators with more profound insights but also significantly contribute to the optimization of LLM applications within the ACT framework.

The fourth component is an artificial intelligence (AI) chatbot logic model, which is responsible for the logical control of the chatbot question flows and detection of the intention of user input, serving as the core to integrate different parts of components to carry out methods such as input and output parsing, data querying and updating, application of Retrieval-Augmented Generation (RAG) 314, few-shot learning and chain-of-thought to stream of data.

As described earlier, the AI logic model may be a pre-trained Natural Language Processing (NLP) model based on the Bidirectional Encoder Representations from Transformers (BERT) architecture that has learned the ACT counseling logic (i.e. an ACT framework 310) and specialized for identifying the user's six psychological flexibility and/or inflexibility process(es) in dialogue texts generated during user interactions with the AI chatbot—the classification module 312. This AI logic model may serve to accurately classify the psychological flexibility and/or inflexibility processes and identify the most prevalent flexibility and/or inflexibility process(es) in the dialogue texts when the user interacts with the AI chatbot, thereby informing which process(es) should be targeted first for process-matched ACT intervention modules through texts, audio records and/or video animations. The AI logic model imitates an ACT interventionist or a counselor in understanding the life circumstances and psychological challenges of the user in a real-person ACT session.

The methods and systems based on the integration of the aforementioned four components have been meticulously designed to cultivate a user-friendly environment, facilitating seamless engagement in conversations, even amidst high-stress situations. As a result, the burden on counselors to conduct exhaustive mental health assessments is markedly diminished, while concurrently maintaining an exceptional user experience.

To enhance the performance of the AI chatbot logic module, the AI logic model is integrated with a third-party NLP model such as OpenAI, which may have the ability to answer general questions. The AI logic model, on the other hand, is designed to handle ACT-related situations. By integrating the AI logic model with OpenAI, the method and systems of the subject invention can improve the overall effectiveness of the AI chatbot logic module by increasing the accuracy and naturalness of the general answering while maintaining the focus on the ACT-related conversations.

Moreover, this integration enhances the smoothness of the conversation flow between questions and answers. As a result, the chatbot logic module of the mental health advisory system of the subject invention provides counseling based on ACT knowledge and enables answering general questions more effectively for improved user experience.

In one exemplary embodiment, a parent of a child with SEN (or a family caregiver) may seek mental health advice and start an initial ACT counseling session by using the Pai.ACT application 300. After completing an initial assessment questionnaire, the user may start “chatting” with the system by interacting with the AI-based chatbot via the chatbot interface using the voice-to-text function. During the initial counseling session, customized questions are provided to the user via the AI chatbot interface, and the user may respond to these questions accordingly, simulating an interaction akin to that with a human ACT counselor. Once the user has finished responding to all questions being asked, the system captures and processes all responses (i.e. the textual inputs 204) provided by the user by classifying all keywords that may be relevant to ACT therapy. Upon completion of the session, the system evaluates the user's mental health status 206, focusing on processes of psychological inflexibility (or flexibility). These include experiential avoidance (or acceptance), cognitive fusion (or defusion), attachment to a conceptualized self (or self-as-context), dominance of a conceptualized past and feared future (or present moment awareness), lack of clarity in values (or values clarification), and patterns of inaction, impulsivity, or avoidance persistence (or committed action). These assessments are visually depicted in a radar chart 400 as shown in FIG. 4. Based on this comprehensive analysis, the system delivers process-matched ACT advice designed to enhance the user's psychological flexibility. Recommendations may include guidance on acceptance, defusion, self-as-context, present moment awareness, values clarification, and committed action. These suggestions are broadly categorized into three actionable strategies: “open up,” “be present,” and “do what matters.” Each strategy targets specific tendencies of psychological inflexibility, such as emotional avoidance, rule-following, or behavioral avoidance, respectively. This tailored advice aims to facilitate meaningful improvements in the user's mental health and behavioral responses.

In accordance with an embodiment of the present invention, there is also provided a method for training the learning network as earlier described, comprising the step of training the learning network with at least one of collecting data, pre-processing data, tokenization, model prediction, model evaluation, and optimizing hyperparameters.

The training may be based on machine learning to build the deep-neural network for analyzing the mental conditions of the users. The machine learning engine, in particular the classification module 312, may be trained with the conversation data between parents and counselors during real counseling sessions, which are real-time videoconferencing-based ACT counseling, and the data may be manually tagged or labeled by the ACT experts. The machine learning method is subsequently harnessed to categorize the dialogues taking place between the user and the AI chatbot, thereby facilitating the method's ability to comprehend the user's mental health condition through the perspectives of the Acceptance and Commitment Therapy (ACT) framework.

The training of the classification module 312 comprises a training flow step and a system testing flow step. The training flow step is provided for training the machine learning processing engine while the system testing flow step is provided for the testing/using of the machine learning processing engine.

Preferably, the training flow unit is configured to perform the following steps.

Data Collection—a large scale of Cantonese text-based data, for example, transcript data, is collected from the therapeutic conversation between an experienced ACT counselor and a parent having a child with special needs during videoconferencing, individual-based ACT counseling.

The transcript data 304 is then categorized and tagged by the ACT experts for determining the six psychological flexibility and/or inflexibility process(es) (a transdiagnostic process that informs Acceptance and Commitment Therapy including experiential avoidance, cognitive fusion, attachment to the conceptualized self, attachment to the conceptualized past and future, lack of values, inaction/impulsivity) and/or three pillars of flexibility and/or inflexibility processes including emotional avoidance, rule-following, behavioral avoidance in dialogue texts generated during user interactions with the AI chatbot. These tagged texts are adopted for the training of retrieval-based Deep-learning engines embedded in the method and systems of the subject invention for improving its accuracy and performance.

Data Pre-Processing—in the step of data pre-processing, certain “stop words” such as “a,” “the,” or “is,” that lack substantial meaning are eliminated, reducing the data noise and highlighting more significant terms such as words that can be categorized as “experiential avoidance”, one of the key psychopathological processes. Further, punctuation marks, symbols, and special characters that do not contribute to the categorization are removed to enhance data cleanliness and streamline the input. Next, data augmentation is performed utilizing techniques such as RoFormerV2, back translation, and libraries such as JioNLP and nlpcda, to enrich the variety and quality of the dataset.

In a preferred embodiment, data pre-processing can include at least the following parts: Stopword Removal: eliminating stopwords based on a custom stopword list; Lowercasing: converting uppercase words to lowercase for uniformity; Punctuation Normalization: transforming full-width punctuation to half-width to ensure consistency; Backtranslation: employing online translation APIs for backtranslation and expanding the training dataset; and Data Augmentation: further enhancing the training dataset through techniques such as paraphrasing or synonym replacement.

Tokenization—the input text is then divided into individual tokens which correspond to the text's smallest meaningful units such as words or sub-words and are associated with a vocabulary. Tokenization with a vocabulary book is designed based on a transformer model such as “hfl/chinese-roberta-wwm-ext”.

Domain Transfer Learning on Pre-Trained Mode—a pre-trained model may be beneficial when addressing specific tasks, such as the classification tasks previously performed. Through the process of domain transfer learning, such a model can be fine-tuned to achieve superior performance within a distinct domain. The domain of interest includes both the Cantonese language and Acceptance and Commitment Therapy (ACT). In this example, as the vast amount of conversational data collected from users in Hong Kong, pre-training of the model using these unannotated datasets is further engaged in. The additional pre-training is configured to refine the model's proficiency in understanding and responding to the unique linguistic and therapeutic characteristics associated with the targeted user population.

Prediction Model Logic Flow Unit—The prediction model logic flow unit is transformer-based, specifically based on the Bidirectional Encoder Representations from Transformers (BERT) type of pre-trained model (Bert and RoBerta).

In one embodiment, the pre-trained model utilizes a Roberta pre-trained model under a transformer model such as “hfl/chinese-roberta-wwm-ext” and wrapped with a model such as “BertForSequenceClassification” which appends two layers ([Dropout(p=0.1, inplace=False), (Linear(in_features=1024, out_features=6, bias=True))]) to adapt to the classification tasks of the subject invention.

Preferably, the learning network may be finetuned by an Adam optimizer with a cosine annealing scheduler and integrating a Retrieval-Augmented Generation (RAG) framework 314 configured to retrieve data from a knowledge database.

In one embodiment, an Adam optimizer from torch.optim.Adam module from torch is selected to optimize the model of the subject invention during model training using the following equations:

$$\begin{matrix}mt = β 1 * m t - 1 + ( 1 - β 1 ) * g t \\ vt = β2 * v t - 1 + ( 1 - β 2 ) * g {t}^{2} \\ \dot{m} t = \frac{m t}{1 - β {1}^{t}} \\ \dot{v} t = \frac{v t}{1 - β {2}^{t}} \\ {θ}_{t} = {θ}_{t - 1} - \frac{γ \cdot \dot{m} t}{\sqrt{\dot{v} t + ϵ}}\end{matrix}$$

gt: gradients calculated via back propagation.
γ: learning rate, preset to be 1e-3.
β1, β2: coefficients for computing running averages of gradient, preset to be 0.9 and 0.99, respectively.
ϵ: to avoid the case when 0 happens in the denominator, preset to be a small value such as 1e-8.
θt: the newly updated parameter after 1 step of optimizing.

Cosine annealing with a warm restart scheduler is adopted to tackle several issues in the training phase, such as being stuck in the local minimum or an instable update in the early phase. Two major factors such as (T0=5, Tmult=2) which contribute to the number of iterations for the first restart, and factors to determine the step to go back to maximum learning rate are utilized. More details of implementation can be found in the CosineAnnealingWarmRestarts module from torch.

$$η t = η min + 1 / 2 * ( η max - η min ) ( 1 + cos ( Tcur * π / Ti ) )$$

η: the learning rate at t iteration.
Tcur: the number of current iterations.
Ti: the number of epochs between two warmup restarts, which can be multiplied by the factor Tmult every time when Tcur=Ti.
T0: the number of iterations for the first restart.

An early stopping technique can be performed with patient number=5 and min_delta=1. Cross entropy loss is selected as the loss function during finetuning the model for the downstream tasks.

When the validation loss exhibits a discernible divergence from the training, it is a strong indicator that the model may be subject to the overfitting issue. To mitigate the potential issue, a technique known as ‘early stopping’ is employed:

$$\begin{matrix}Initiate min ValLoss = 0 , Counter = 0 \\ while True : \\ if valLoss > min ValLoss + Δ : \\ Counter ++ \\ if Counter > patience : \\ break \\ else if valLoss < min ValLoss : Counter = 0\end{matrix}$$

Cross entropy loss is selected to calculate the loss between input logits and target

$$CrossEntropyLoss = - {\sum }_{i = 1}^{C} {p}_{i} log ( {q}_{i} )$$

C: a number of classes
pᵢ: the truth values
qᵢ: the predicted logits

The built network may be evaluated, e.g. by assessing diagnostic ability of a classifier as a discrimination threshold is varied by a Receiver Operating Characteristic (ROC) method. More preferably, the system may be further evaluated by refining the classifier's accuracy in diagnosing and identifying psychological flexibility and/or inflexibility processes by employing a Confusion Matrix, providing a comprehensive view of overall performance of the model and determining whether an imbalance class is mostly predicted as a major class.

To further refine the accuracy of the classification module 312 in identifying psychological flexibility and/or inflexibility processes, a Confusion Matrix 316 may be employed, in which each row represents the actual class, and each column represents the predicted class. The diagonal of the matrix 316 represents the correct prediction of each class, while the off-diagonal elements indicate where the misclassification occurs. This matrix 316 provides a comprehensive view of the overall performance of the model. By examining the elements in the matrix 316, whether an imbalanced class is mostly predicted as a major class is determined. This matrix 316 also provides a detailed breakdown of the classifier's performance and can be expanded as True Positives, False Positives, True Negatives, and False Negatives with the means of one-vs-one or one-vs-rest. By analyzing these categories, the classifier's precision, recall, and F1 scores can provide nuanced insights into its ability to correctly identify and categorize psychological flexibility and/or inflexibility processes. The analysis also discusses the granularity of the target variable in the classification task with a design of classification among three categories, and classification among six categories.

The accuracy, micro F1 score, or macro F1 score are adopted, since the tasks are classified by single-label classification instead of multi-label classification. The micro F1 score is defined by the accuracy equation below:

$$\begin{matrix}Accuracy or micro F 1 = \frac{T P}{T P + F N} \\ macro F 1 = \frac{F 1 score of each class}{Num of class}\end{matrix}$$

TP: true positive cases
FN: false negative cases
Precision: ratio of true positive predictions to the sum of true positive and false positive predictions. It measures the correctness achieved in the positive predictions.

$$Precision = \frac{T P}{T P + F P}$$

where TP is true positive cases and FP is false positive cases.
Recall/Sensitivity: a ratio of true positive predictions to the sum of true positive and false negative predictions. It measures the ability of the classifier to find all the positive instances.

$$Precision = \frac{T P}{T P + F N}$$

where TP is true positive cases and FN is false negative cases.
Specificity: a ratio of true negative predictions to the sum of true negative and false positive predictions. It measures the ability of the classifier to correctly identify negative instances.

$$Precision = \frac{T N}{T N + F P}$$

TN: true negative cases
FP: false positive cases
NPV (Negative Predicted Value): a ratio of true negative predictions to the sum of true negative and false negative predictions. It measures the correctness achieved in the negative predictions.

$$Precision = \frac{T N}{T N + F N}$$

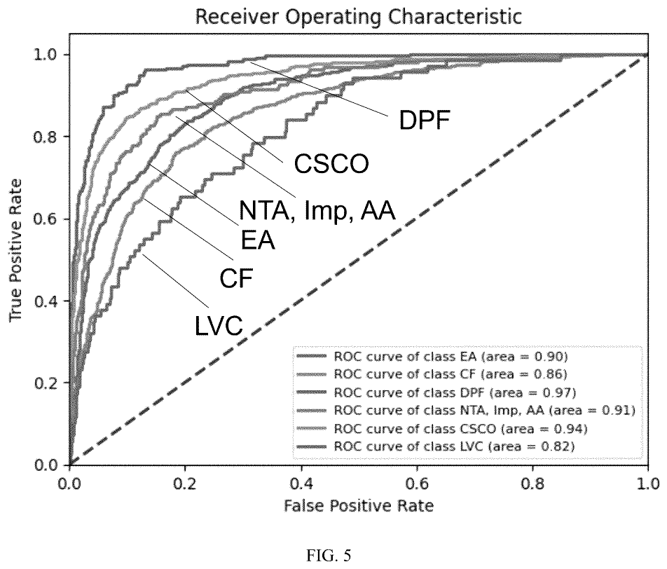

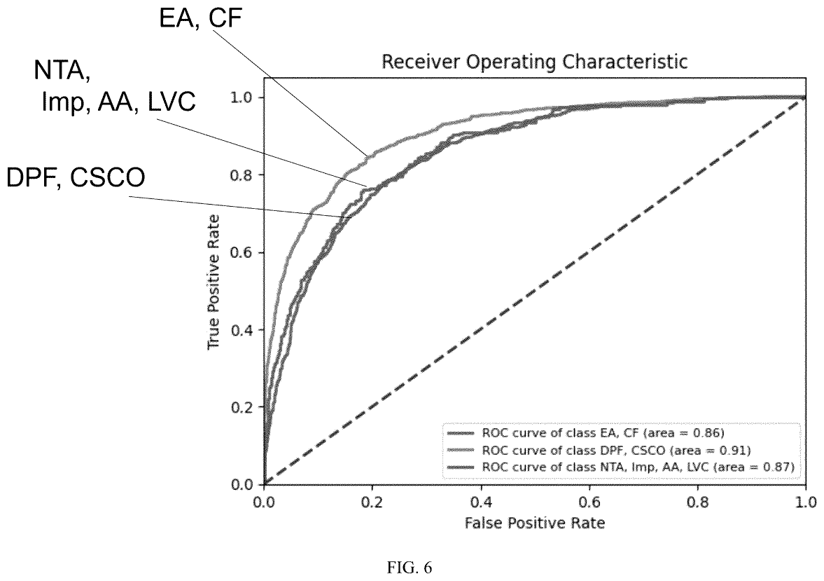

TN: true negative cases
FN: false negative cases
Confusion Matrix: In multi-class classification, the confusion matrix 316 has dimensions N times N, where N is the number of classes.
The rows represent the actual classes
The columns represent the predicted classes
The diagonal from the top left to the bottom-right represents the true positive counts for each class
The off diagonal elements represent the misclassifications
Receiver Operating Characteristic (ROC) Curve: Referring to FIG. 5 and FIG. 6, the ROC curve illustrates the diagnostic ability of a classifier as its discrimination threshold is varied. The plot is created by plotting the True Positive Rate (TPR/Recall/Sensitivity) against the False Positive Rate (FPR) at various threshold settings. A one-vs-rest or one-vs-one and then averaging approach is adopted to extend to multi-class classification scenarios.

$$\begin{matrix}TPR = \frac{T P}{T P + F N} \\ FPR = \frac{F P}{T N + F P}\end{matrix}$$

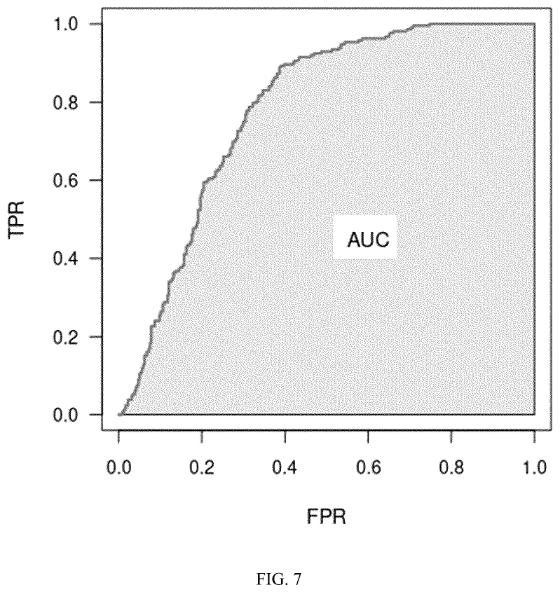

One-vs-all approach
1. For each class: Binarize the prediction n (determined 1 positive and mark the rest as a negative label).
2. Select the threshold based on each prediction value from the dataset.
3. Calculate TPR and FPR for each class and draw the ROC curve.
One-vs-one approach
1. Select arbitrary 2 class and determine 1 as positive and mark the other as negative label.
2. Select the threshold based on each prediction value from the dataset.
3. Calculate TPR and FPR and draw the ROC curve.
Area under the ROC Curve (AUC) scores: As shown in FIG. 7, the AUC scores quantify the area under the ROC curve which summarizes the overall performance of the classifier across all possible area/threshold settings. An AUC of 0.5 suggests no discrimination (similar to random guessing), while an AUC of 1.0 signifies perfect discrimination.

Hyperparameters are configuration settings that are not learned during the training process but directly impact the performance of the model. Hyperparameter tuning comprises systematically adjusting these settings to find the optimal combination that yields the best performance.

After the system is built and evaluated, the system may be tested in the system testing flow step, which includes at least the steps of segmenting sentences, pre-processing data, predicting, and post-processing, further explained as follows: Sentence Segmentation—multiple predicting inputs of different ROI may be created from the paragraphs that are collected from the AI chatbot with respect to the n-gram basis.
Sentence Segmentation Flow
1. define n=3, segment list=[ . . . ]
2. segment sentences according to the segment list→[list of sentence pieces in order]
3. Generate multiple input based on different ROI
For example, n=2: [aaa, bbb, ccc]→[[aaa], [bbb], [ccc], [aaa bbb], [bbb, ccc]] Return 5 possible input for prediction

Data Pre-Processing—stop words that do not carry significant meaning such as “a,” “the,” or “is” are removed to reduce noise in the data to focus on more important words.

Moreover, punctuation marks, symbols, and special characters that do not contribute to the categorization are removed to clean the data and simplify the input.

Prediction Model Inferencing—the generated batch of inputs is fed into the prediction model of the subject invention and outputs probability distributions over targeted categories.

Postprocessing—the prediction result must surpass the confidence threshold to be considered valid and to exclude the “none of the above” scenarios.
A confidence threshold for probability distribution: if the probability is smaller than the threshold, the sentence is considered to belong to neither of the 6 categories:
1. probability distribution p=[x, y, z, . . . ]
2. return highest k probability with a parameter topK
3. Sort the probability in desc order
4. For i in probability list:
   if probability>Threshold:
      return prediction
   else return none of above
Non-maximum suppression can be performed over these batches of input and the calculation of Intersection over Union (IoU) is required.
Non maximum suppression
1. Define an empty list Bₙₘₛ=Φ
2. sort(segment process output) in desc order based on prediction→ [symbol]
3. for Sᵢ in [symbol] :
   for sⱼ in the remaining [symbol] :
      if IOU(sᵢ, sⱼ)>IOU_{threshold}:
         remove Sⱼ from [symbol]
4. Return final [symbol] with remaining elements

$$IOU calculation = 2 \cdot \frac{longest common sequence ( s 1 , s 2 )}{sum ( {length}_{s 1} + {length}_{s 2} )}$$

The final remaining outcomes can be sent back by the method and systems of the subject invention for further analysis.

In one embodiment, the NLP Models, particularly the language model such as “hfl chinese-roberta-wwm-ext”, can be fine-tuned and customized by utilizing the collected data. The process involves adapting the pre-trained model to better understand the unique vocabulary, expressions, and conversational styles relevant to the parents of children with special needs.

In another embodiment, domain-specific language proficiency can be enhanced. In particular, the system's understanding of domain-specific terminology and language patterns related to Acceptance and Commitment Therapy (ACT) and the challenges faced by the parents of children with special needs are provided. This process may involve building a custom vocabulary or embedding specific domain knowledge of ACT into the model.

In another embodiment, the scalability and performance of the subject invention can be optimized by improving the entire backend and frontend method and systems with respect to the architecture and infrastructure for handling increased user demands.

In another embodiment, long-term data collection and efficacy testing can be enhanced by continually collecting user data to refine machine learning models. Moreover, user interactions with the chatbot and other Pai.ACT features, outcomes (for example, 6 to 12-month follow-up on mental health outcomes), and user feedback can be analyzed to refine Pai.ACT.

In one example evaluation process, the “User Input” component may serve as the primary interface for data acquisition, capturing information either via the AI chatbot interactions or specific service sessions. After data is collected, the module channels the acquired data to the AI chatbot, which functions as the central processing unit of the system. Within this module, real-time computational operations are performed to generate contextually appropriate prompts and responses. Central to the performance of the AI chatbot is the Knowledge Database, a repository containing essential data that enables effective implementation of Retrieval-Augmented Generation (RAG) 314. The database 304 stores quantitative and qualitative metrics related to user behavior, including but not limited to indicators of self-harm tendencies, conversational engagement levels, duration of interactions, and average response lengths.

Furthermore, the utilization of question summarization algorithms enhances the LLM's contextual comprehension, facilitating more individualized responses. The Knowledge Database is dynamic and updated continually by the AI chatbot. During data processing, the AI employs a predetermined prompt template, calibrated to align with a chain-of-thought paradigm. Upon completion of the LLM's processing phase, the generated output is subjected to an evaluation by a final metric to ascertain its relevance and appropriateness.

In addition, to enhance the system's robustness, irrelevant handling 212 has been integrated. The irrelevant handling process is activated when the LLM detects potential incongruities in contextual relevance. It incorporates features such as initiating auxiliary conversational sessions to gather more relevant user data, displaying statistical metrics related to the relevancy performance across different LLM iterations, and suggesting proactive user interactions to refine the acquisition of pertinent information. As a result, a layer of system adaptability is added and more meaningful engagement with users is ensured.

It was observed that the performance assessment approach for the system hinges on two pivotal metrics. The initial metric focuses on the precision of irrelevant response detection, rooted in data previously collected from the AI chatbot or services session. Calculating this metric may necessitate minimal labeling undertakings by operators. The secondary metric pertains to the efficacy of the chatbot's feature that proactively prompts additional information upon receiving irrelevant user inputs. Subsequent user responses are analyzed to ascertain the success rate of the AI chatbot's proactive data collection in the face of irrelevant inputs. As more data is accumulated, refinement of the system design regarding the RAG system and prompt design are performed. User engagement, diagnostic accuracy, and algorithmic fidelity of the advanced system are calculated.

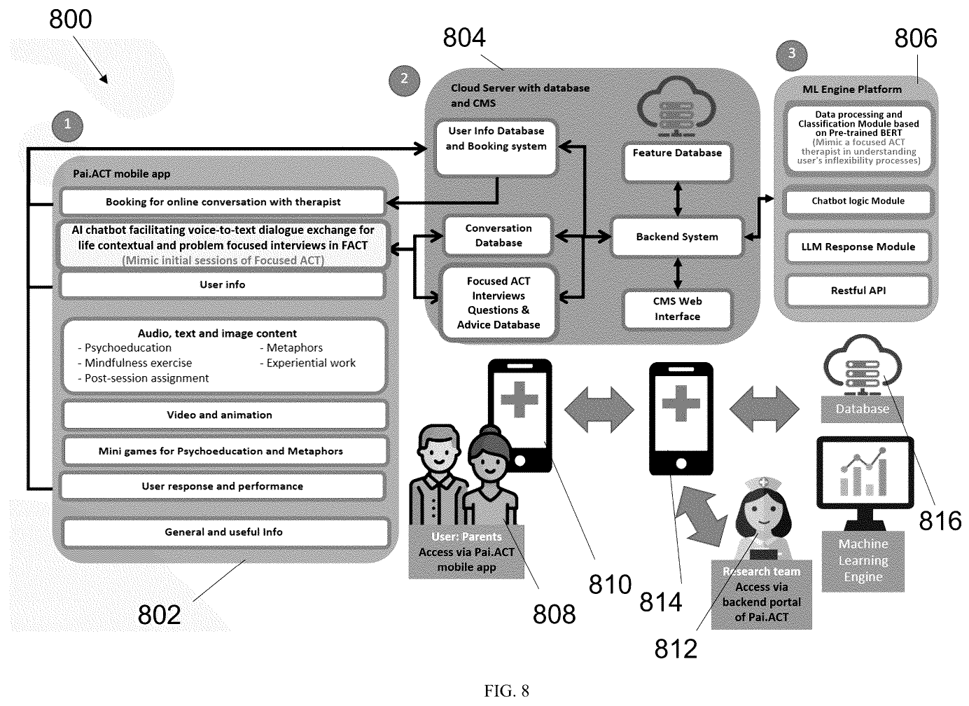

With reference to FIG. 8, the core components of the system 800 and accordingly their example applications are illustrated. In this example, (1) a mobile application 802 named ‘Pai.ACT’ may be provided for data collection and mental health servicing for users, where the application 802 may be installed on user devices 810; (2) Cloud Service and database with content management system (CMS) 804 and (3) a machine learning engine platform 806, including modules that support the chatbot infrastructures mentioned earlier. The mobile application 802 is used by users 808, such as parents of children with SEN, collects information through a questionnaire and conversations with the AI chatbot system and also provides functionality such as booking appointments for meetings with counselors and performing interactive actions for mental health servicing. The Cloud Service and database with CMS provide not only an interface for the operator 812, such as an ACT research team member, to import data, but also a platform to visualize the analysis of the user's mental state, history of chat and other information with ease, via an operator device 814. The machine learning component 806 serves as an engine to power up the previously mentioned chatbot infrastructure by hosting an AI chatbot and a classification model using BERT and provides the functionality with a restful API to the backend. In addition, the machine learning may be dynamically trained by updated database 816 incorporating chat histories and/or operators' imported data upon completion of consultation sessions initiated by users 808.

These embodiments may be advantageous in that, the system may be operable to provide a retrieval-based mental health advisory platform, having acquired the domain expertise of Acceptance and Commitment Therapy (ACT) counseling logic. Through the voice-to-text module embedded in the AI chatbot, it is designed to autonomously evaluate the fundamental psychopathological processes experienced by parents of children with special needs, encompassing six psychological flexibility and/or six psychological inflexibility process(es) (a transdiagnostic process that informs ACT including experiential avoidance, cognitive fusion, attachment to the conceptualized self, lack of present-moment awareness, and inaction/impulsivity in the pursuit of a valued goal) and/or three pillars of flexibility and/or three pillars of inflexibility processes including emotional avoidance, rule-following, behavioral avoidance.

Advantageously, the method and system then offer treatment interventions aligned with the ACT principles. By furnishing real-time, early consultancy services, spanning assessment, intervention, and follow-up, the method and systems of the subject invention are configured to validate the perceived effectiveness and utility for improving the mental well-being of parents navigating the challenges of caring for their children with special needs. An integral outcome of the method and systems of the subject invention resides in its intrinsic capacity to ameliorate the operational burden borne by frontline mental health practitioners.

In addition, through real-time, personalized support via an intuitive mobile application, the method and systems of the subject invention have the capacity to alleviate emotional burdens, enhance parental well-being, and improve the overall quality of life for the parents and their children. Advantageously, by providing 24/7 mental health support to Chinese-speaking parents, Pai.ACT can provide a one-stop, effective, highly accessible and low-cost mental health advisory system and method to all Chinese-speaking parents of children with special needs, ultimately enhancing the overall well-being of these vulnerable families. Additionally, where the system detects interrupting conditions, such as the threat to the well-being of the user or family members from the dialogue (e.g. self-harm, the physical harming of others, etc.) or potentially urgent situations, or request for specific services or information, the system may also be able to detect these interrupting conditions, and direct assistance to the user (e.g. referral to information, connection or scheduling of appointments with medical professionals, or the direct contact with emergency services or social workers).

Moreover, the system also provides the following advantages in different applications: Extended service and subscriptions: Collaborators and NGO partners collaborating with the inventors possess the opportunity to avail themselves of Pai.ACT's comprehensive maintenance services. These services encompass individualized ACT counseling and access to professional ACT training subscriptions tailored for paraprofessionals. Pai.ACT mobile application and its related deep-learning training models strategically offer a spectrum of flexible application/pricing models, encompassing subscription-based plans, one-time fee options, freemium models featuring in-app purchases, and pay-per-service structures. This multifaceted pricing strategy not only guarantees user satisfaction but also serves as a revenue generation mechanism for the platform.

Adaptability and re-training: The inherent adaptability and re-training capabilities of the system are facilitated by user-chatbot interactions via Pai.ACT, and in some cases, through engagement with ACT counselors, underscore its remarkable scalability. This inherent flexibility strategically situates Pai.ACT as a multifaceted solution with broad applicability across diverse population segments, thereby significantly expanding its market potential.

Enhanced AI chatbot capabilities: The AI chatbot in Pai.ACT exhibits an amplified capacity to recalibrate its questioning logic upon encountering irrelevant responses. It possesses the autonomy to actively engage and interact with users to procure more pertinent information, which subsequently augments downstream tasks, such as the detection task.

Comprehensive data visualization: A salient feature of the system is its capability to display statistics concerning query-response pairs and the proclivity of different users towards irrelevant responses. This method provides system operators with a clear snapshot of current user metrics and the quality of ongoing conversation sessions. Such insights not only empower operators with a deeper understanding but also contribute richly to harnessing LLM in the ACT domain.

Global expansion and cross-border collaborations: Leveraging its proficiency in the Chinese languages, including Cantonese, Pai.ACT stands poised to offer support to Cantonese-speaking families around the globe, particularly those with children having special needs. Pai.ACT's adaptable framework, designed to tackle psychological challenges using ACT counseling techniques, can be seamlessly tailored to suit diverse linguistic and cultural environments, thus extending its reach and unlocking new market opportunities worldwide.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components, and data files assisting in the performance of specific functions, the skilled person will understand that the functionality of the software application may be distributed across a number of routines, objects, or components to achieve the same functionality desired herein.

It will also be appreciated that where the methods and systems of the present invention are either wholly implemented by computing systems or partly implemented by computing systems then any appropriate computing system architecture may be utilized. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

We claim:

1. A method for providing mental health advice, comprising the step of:
receiving textual input from a user in a counseling session;
processing the textual input by applying a mental health condition relationship to the textual input to identify the mental health status of the user; and
providing an output associated with the mental health status of the user.

2. The method in accordance with claim 1, wherein the mental health condition relationship is trained by a deep-learning network.

3. The method in accordance with claim 2, wherein the deep-learning network is a deep-neural network arranged to identify the mental health status based on the received textual input being labeled by the deep-neural network.

4. The method in accordance with claim 3, wherein the textual input includes dialogues provided by the user in response to one or more predetermined questions in a question bank based on Acceptance and Commitment Therapy (ACT) knowledge in the counseling session.

5. The method in accordance with claim 4, wherein the question bank is configured to imitate life-contextual and problem-focused interviews between a counselor and the user at a real-person ACT counseling session for an understanding of the user's mental health condition in terms of status or processes of psychological inflexibility and/or psychological flexibility.

6. The method in accordance with claim 5, further comprises the step of providing an artificial intelligence (AI) chatbot interface arranged to facilitate asking the user one or more predetermined questions and receiving the textual input from the user in the consultation session.

7. The method in accordance with claim 6, wherein one or more predetermined questions are provided to the user in the form of a questionnaire and/or one or more chat dialogues.

8. The method in accordance with claim 4, wherein the question bank is generated upon an initial real-person ACT counseling session between the counselor and the user.

9. The method in accordance with claim 4, wherein the question bank is generated based on a self-reported mental health assessment completed by the user, wherein the self-reported mental health assessment is associated with an evaluation of anxiety symptoms, depressive symptoms, stress, psychological inflexibility or psychological flexibility of the user.

10. The method in accordance with claim 6, further comprises the step of detecting at least one irrelevant response provided by the user in the consultation session, and obtaining confirmative responses from the user.

11. The method in accordance with claim 10, wherein the AI chatbot interface is arranged to adjust lines of inquiry upon determination of non-pertinent user responses, if a predetermined number of irrelevant responses is detected.

12. The method in accordance with claim 11, wherein the AI chatbot interface is arranged to facilitate providing an additional round of questions to the user if non-pertinent user responses are detected, to obtain the confirmative responses from the user for an accurate determination of the mental health status of the user.

13. The method in accordance with claim 12, wherein the AI chatbot interface is further arranged to autonomously initiate and sustain engagement with the user and steer conversations to extract information of greater relevance for identifying the mental health status of the user.

14. The method in accordance with claim 11, wherein the output includes an occurrence of irrelevancy in the consultation session.

15. The method in accordance with claim 6, wherein the AI chatbot interface is further supported by an external Large Language Model (LLM) processing engine arranged to enhance the generation of contextually relevant responses to facilitate interacting with the user via the AI chatbot interface.

16. The method in accordance with claim 1, further comprises the step of pre-processing the textual input before applying the mental health condition relationship to the textual input for identifying the mental health status of the user.

17. The method in accordance with claim 16, wherein pre-processing of the textual input includes processing the textual input with at least one of removing stopwords, lowercasing, punctuation normalizing, back translation and data augmentation.

18. The method in accordance with claim 1, wherein the mental health status includes the psychological inflexibility or psychological flexibility of the user.

19. The method in accordance with claim 18, wherein the mental health status is associated with the following processes of psychological inflexibility or psychological flexibility:
experiential avoidance or acceptance;
cognitive fusion or cognitive defusion;
conceptualized past and fear of future or present-moment awareness;
attachment to conceptualized self or self-as-context;
lack of values clarity or values clarifications; and
inaction, impulsivity, avoidance persistence or committed action.

20. The method in accordance with claim 4, wherein the deep-learning network is a pre-trained Natural Language Processing (NLP) model based on Bidirectional Encoder Representations from Transformers (BERT) architecture, including Robustly Optimized BERT Pretraining Approach (RoBERTa) architecture, that has learned ACT counseling logic.

21. A method for training a deep-learning network in accordance with claim 20, comprises the step of training the deep-learning network with at least one of collecting data, pre-processing data, tokenization, model prediction, model evaluation, and optimizing hyperparameters.

22. The method in accordance with claim 21, further comprises the step of finetuning the deep-learning network by an Adam optimizer with a cosine annealing scheduler and integrating a Retrieval-Augmented Generation (RAG) framework configured to retrieve data from a knowledge database.

23. The method in accordance with claim 22, wherein the knowledge database is dynamic and consistently updated by chat statistics and questions summary.

24. The method in accordance with claim 22, wherein the deep-learning network is evaluated by assessing the diagnostic ability of a classifier as a discrimination threshold is varied by a Receiver Operating Characteristic (ROC) method.

25. The method in accordance with claim 24, wherein the deep-learning network is further evaluated by refining the classifier's accuracy in diagnosing and identifying psychological flexibility and/or inflexibility processes by employing a Confusion Matrix, providing a comprehensive view of the overall performance of the model and determining whether an imbalance class is mostly predicted as a major class.

26. The method in accordance with claim 21, wherein further comprises the step of testing the deep-learning network with at least one of the segmenting sentences, pre-processing data, predicting, and post-processing.

27. A deep-learning based mental health advisory system using Acceptance and Commitment Therapy (ACT), comprising:
an AI chatbot interface in accordance with claim 6 provided on a user device, wherein the AI chatbot interface is supported by the deep-learning network; and
a cloud service and database with a content management system (CMS).

28. The system of claim 27, wherein the user device is arranged to collect the textual input through a questionnaire and/or conversations via the AI chatbot interface and to provide functionality including one or more of the followings:
process-matched ACT interventions in accordance with one or more processes of psychological inflexibility or psychological flexibility as identified and diagnosed;
tailored stepped-care mental health interventions based on the ACT principles; and
booking appointments for meetings with counselors and performing interactive actions for mental health servicing.

29. The system of claim 27, further comprises an operator interface supported by the Cloud Service and the database with the CMS, wherein the operator interface is arranged to facilitate importing data and visualizing analysis of the mental health status of the user and chat history.

30. The system of claim 27, further comprising a portal arranged to display the output associated with the mental health status of the user and/or statistics with respect to detected irrelevant contents and frequency/occurrences of irrelevancy.
